(19) United States
(12) Patent Application Publication
(10) Pub. No.: US 2024/0173515 A1
(43) Pub. Date: May 30, 2024

(54) AUDIO GENERATION DEVICE AND METHOD FOR BRAIN DYNAMICS AUDIO STIMULATION

(71) Applicant: Ming-Chung HO, Kaohsiung City (TW)

(72) Inventor: Ming-Chung HO, Kaohsiung City (TW)

(21) Appl. No.: 18/380,694

(22) Filed: Oct. 17, 2023

(30) Foreign Application Priority Data
Nov. 24, 2022 (TW) 111145096

(51) Int. Cl. A61M 21/02; A61B 5/00; A61B 5/374; A61B 5/375; A61B 5/38; G10K 15/02; G16H 20/70

(52) U.S. Cl. CPC A61M 21/02; A61B 5/374; A61B 5/375; A61B 5/38; A61B 5/4064; A61B 5/4809; A61B 5/4812; A61B 5/4836; A61B 5/4857; A61B 5/7257; A61B 5/7278; A61B 5/7282; G10K 15/02; G16H 20/70; A61M 2021/0027

(57) ABSTRACT

An audio generation device for brain dynamics audio stimulation includes an audio playing device, a brainwave capturing device, and a computing device. The audio playing device sequentially outputs multiple audio signals to give a subject sound stimulation with various audio frequencies. The brainwave capturing device captures brainwave signals from the subject. The computing device analyzes the pairwise dynamics correlation of the channel signals of the brainwave signal to obtain the correlation of the transfer direction associativity in response to the brain stimulation, synthesizes a test audio according to at least one audio frequency corresponding to a target channel, and makes the test audio as an output audio when the target channel belongs to the high-correlation response channel of the brainwave signal that the subject generates in response to listen to the test audio. An audio generation method for brain dynamics audio stimulation is also provided.

### CROSS-REFERENCE TO RELATED APPLICATION

This non-provisional application claims priority under 35 U.S.C. § 119(a) to Patent Application No. 111145096 filed in Taiwan, R.O.C. on Nov. 24, 2022, the entire contents of which are hereby incorporated by reference.

### BACKGROUND

### Technical Field

The present disclosure relates to an audio generation technology, and in particular, to an audio generation device and method for brain dynamics audio simulation.

### Related Art

In recent years, with the rapid development of living pace, general symptoms of insomnia, anxiety, depression, and great psychological stress of people gradually aggravate, and particularly, problems related to the brain, such as migraine and dementia, are more serious. At present, methods of transcranial stimulation for brain stimulation are currently developed, and the transcranial stimulation are broadly divided into electrical stimulation and magnetic stimulation.

Transcranial electrical stimulation operates on the principle of placing electrode pads on the scalp. Then, electrodes emit weak current to the brain. Transcranial magnetic stimulation is the application of transient magnetic pulses to the brain that cause magnetic fields to travel through the skull, stimulating brain tissue to produce changes in neuron activity.

However, whether the transcranial electrical stimulation or the transcranial magnetic stimulation is essentially application to people with simple electrical or magnetic signals that are easily controlled externally, and after the signals are applied to the brain, the activity changes of brain neurons cannot be analyzed in a complete theory, so that the response performance of the brain cannot be expected or guaranteed.

### SUMMARY

An embodiment of the present disclosure provides an audio generation device for brain dynamics audio stimulation, and the device includes an audio playing device, a brainwave capturing device, and a computing device. The audio playing device is configured to sequentially output multiple audio signals with multiple audio frequencies to give a subject sound stimulation. The brainwave capturing device is configured to capture a first brainwave signal from the subject, and the first brainwave signal includes a plurality of first channel signals. The computing device is coupled with the brainwave capturing device and configured to analyze pairwise correlation in the first channel signals to obtain a plurality of high-correlation first response channels corresponding to multiple different audio frequencies, and synthesize a test audio according to at least one audio frequency corresponding to a target channel. The target channel is selected from the first response channels and corresponds to a target stimulation area. The audio playing device is further configured to output the test audio. The brainwave capturing device is further configured to capture a second brainwave signal from the subject. The computing device is further configured to analyze pairwise correlation in second channel signals to obtain a plurality of high-correlation second response channels. The second response channels include the target channel, and the test audio serves as an output audio.

An embodiment of the present disclosure provides an audio generation method for brain dynamics audio stimulation, which includes: (a) outputting an audio signal with an audio frequency via an audio playing device to give a subject sound stimulation; (b) capturing a brainwave signal from the subject via a brainwave capturing device, the brainwave signal including a plurality of channel signals; (c) analyzing pairwise correlation in the channel signals via a computing device to obtain a plurality of high-correlation response channels; (d) repeatedly executing steps (a) to (c) to obtain response channels corresponding to multiple different audio frequencies; (e) determining a target channel corresponding to a target stimulation area from the response channels by the computing device; and (f) synthesizing a test audio by the computing device according to at least one audio frequency corresponding to the target channel, outputting the test audio via the audio playing device, and executing steps (b) and (c), and making the test audio as an output audio when the target channel belongs to the response channels.

In summary, according to the audio generation device and method for brain dynamics audio stimulation provided by some embodiments of the present disclosure, the audio signal for specific stimulation purpose can be generated efficiently, and the generated audio signal can be outputted to stimulate the brain efficiently.

### BRIEF DESCRIPTION OF THE DRAWINGS

FIG. 1 is a schematic structural diagram of an audio generation device for brain dynamics audio stimulation according to an embodiment of the present disclosure.

FIG. 2 is a flowchart of an audio generation method for brain dynamics audio stimulation according to an embodiment of the present disclosure.

FIG. 3 is a spectral energy diagram of brainwaves of a channel signal (auditory area T6) at different time intervals according to an embodiment of the present disclosure.

FIG. 4 is a spectral energy diagram of brainwaves of another channel signal (visual area O1) at different time intervals according to an embodiment of the present disclosure.

FIG. 5 is a distribution diagram of electrodes according to an embodiment of the present disclosure.

FIG. 6 is a schematic diagram of sleep levels of a subject subjected to audio stimulation according to an embodiment of the present disclosure.

### DETAILED DESCRIPTION

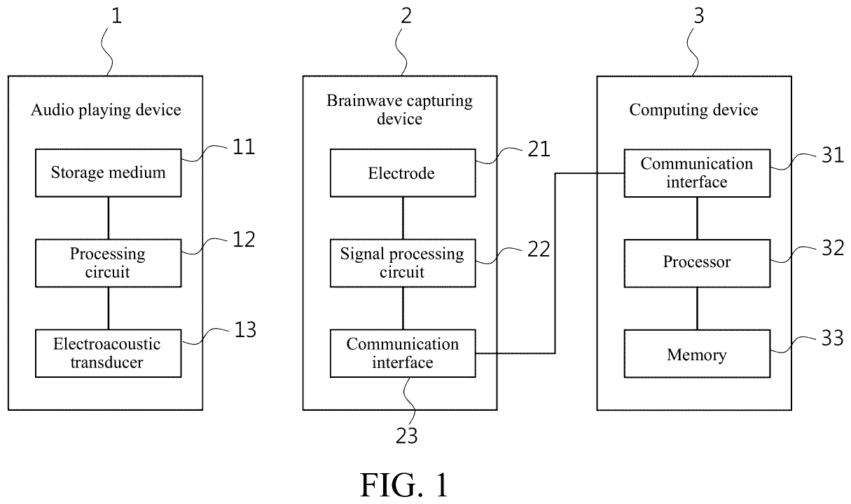

Referring to FIG. 1, which is a schematic structural diagram of an audio generation device for brain dynamics audio stimulation according to an embodiment of the present disclosure. The audio generation device for brain dynamics audio stimulation includes an audio playing device 1, a brainwave capturing device 2, and a computing device 3.

The audio playing device 1 includes a storage medium 11, a processing circuit 12, and an electroacoustic transducer 13. The processing circuit 12 is coupled between the storage medium 11 and the electroacoustic transducer 13. The storage medium 11 is a non-transitory storage medium (such as a hard disk, a flash memory, and an optical disk) that stores audio files. The processing circuit 12 may be a processor, an audio driving circuit, and/or a digital signal processor, and is configured to decode the audio files and process audio signals, and transmit the audio signals to the electroacoustic transducer 13 for output. The electroacoustic transducer 13 may be, for example, an earphone, a horn, a loudspeaker, and the like.

The brainwave capturing device 2 includes a plurality of electrodes 21 (for example, 32, 64, 128, and 256 electrodes), a signal processing circuit 22, and a communication interface 23. The signal processing circuit 22 is coupled between the plurality of electrodes 21 and the communication interface 23. The electrodes 21 are configured to capture brainwave signals including a plurality of channel signals. The signal processing circuit 22 may be a processor, an analog signal processing circuit, and/or a digital signal, is configured to process the channel signals (such as analog-to-digital conversion, signal amplification, and filtering), and output the processed channel signals to the computing device 3 through the communication interface 23. The communication interface 23 may be a wired communication interface (such as RS232, USB, and wired network) or a wireless communication interface (such as Bluetooth, and Wi-Fi).

The computing device 3 may be, for example, a personal computer, a notebook computer, a server, and the like, and includes a communication interface 31, a processor 32, a memory 33, and the like. The processor 32 is coupled between the communication interface 31 and the memory 33. The communication interface 31 is configured to be in communication connection with the communication interface 23 of the brainwave capturing device 2 to obtain brainwave signals. The processor 32 is configured to analyze the channel signals in the brainwave signals, generate an output audio according to the analysis result, and store the output audio in the memory 33.

In some embodiments, the audio playing device 1 is integrated, in whole or in part, with the computing device 3.

In some embodiments, the brainwave capturing device 2 is integrated, in whole or in part, with the computing device 3.

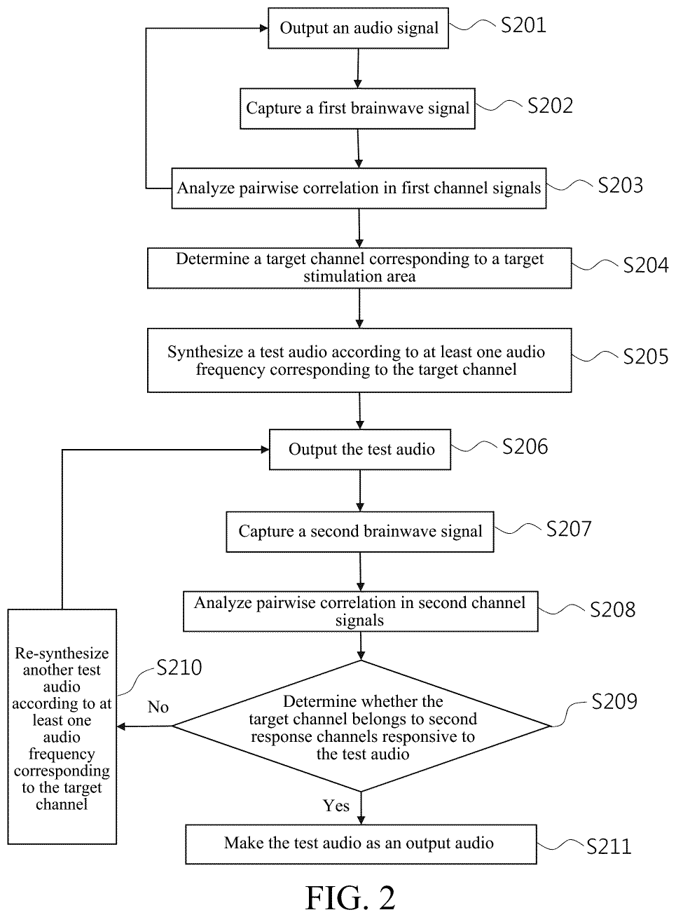

Referring to FIG. 2, which is a flowchart of an audio generation method for brain dynamics audio stimulation according to an embodiment of the present disclosure. Firstly, step S201 to step S203 are repeated for a plurality of times in multiple cycles, where the audio frequency of the audio signal outputted in each execution cycle is different. In step S201, an audio signal with an audio frequency is outputted by an audio playing device 1 to give a subject sound stimulation. In step S202, a brainwave signal (hereinafter referred to as a first brainwave signal) is captured from the subject via a brainwave capturing device 2, and the first brainwave signal is used for subsequent analysis of the response of the subject to the audio stimulation. In step S203, pairwise correlation in a plurality of channel signals (hereinafter referred to as first channel signals) in the first brainwave signal is analyzed via a computing device 3, that is, the computing device analyzes correlation of any two of the first channel signals. The high-correlation channels are considered as channels having a response (hereinafter referred to as first response channels). After step S201 to step S203 are repeatedly executed in multiple cycles, first response channels corresponding to multiple different audio frequencies may be obtained. In some embodiments, the audio frequency used is selected from a range of 1 Hz to 100 Hz.

By finding channels that have a response to each other in terms of audio frequency, correlation between the effect of the audio stimulation and conduction of audio stimulation in brain areas may be determined. In step S204, the computing device 3 determines a channel (hereinafter referred to as a target channel) corresponding to a target brain stimulation area (hereinafter referred to as a target stimulation area) from the first response channels. The target stimulation area may be selected depending on a stimulation purpose to be achieved.

Then, in step S205, the computing device 3 synthesizes a test audio according to at least one audio frequency corresponding to the target channel. In other words, after the above steps S201 to S203 are repeated in multiple cycles, it can be known which audio frequency or audio frequencies corresponding to the target channel (a certain selected first response channel) has or have a response. Therefore, in step S205, one or more of the audio frequencies having a response are selected to be synthesized into the test audio. In other words, the test audio includes one or more audio frequencies that are corresponding to the target channel and have a response.

In step S206, the test audio is outputted via the audio playing device 1, and then step S207 and step S208 are executed. Step S207 is similar to the previous step S202, in this step, a brainwave signal (a second brainwave signal) is captured from the subject via the brainwave capturing device 2. Step S208 is similar to the previous step S203, in this step, pairwise dynamics correlation in multiple channel signals (second channel signals) in the brainwave signals is analyzed via the computing device 3 to obtain a plurality of high-correlation response channels (second response channels), which show the correlation of the transfer direction associativity in response to the brain stimulation. In step S209, it is determined whether the target channel belongs to the second response channel that is responsive to the test audio. If the target channel belongs to the second response channel that is responsive to the test audio, the test audio is made as an output audio (step S210). If the target channel does not belong to the second response channel that is responsive to the test audio, step S211 is executed to re-synthesize another test audio according to at least one audio frequency corresponding to the target channel (that is, re-synthesize another test audio composed of at least one audio frequency corresponding to the target channel and not overlapping with the previously synthesized test audio), and step S206 is executed to perform the test again until a suitable audio is found.

By the mode, a user may be subjected to audio stimulation by the generated output audio for the stimulation purpose. The rationale for this is that sleep is a mechanism by which the brain resets at rest, when the user sleeps, the brain is in an initial energy ground state. When the brain works, the spectral energy of the brain may be increased. When the user is subjected to the audio stimulation, the brain certainly has a feedback effect, but the brain has different strong and weak responses due to different functions of the areas of the brain. The area with stronger response is indicated as the area of signal transmission. By the areas where these signals resonate (the signals have high correlation), a signal transmission path may be found out. When a signal transmission path (representing zero dispersion of stimulation signals) capable of transmitting signals to the area to be acted on is found, it indicates that the stimulation can achieve the purpose of the desired stimulation. According to the present disclosure, a brain dynamics theory is used for performing directional time series analysis, determining life sustaining and processing the signals; and then, the brain dynamics audio is formed by applying information flow direction brainwave zero dispersion nonlinear resonance analysis.

Theories and inferences of the brain dynamics theory are established as follows: (1) a first theory: sleep is a mechanism by which the brain resets at rest, at the moment, the brain is in an initial energetic ground state. (2) A first inference: when the brain is in a resting and life sustaining state, the spectral energy of brainwaves is in a ground state; and when the brain works in an operation state, the spectral energy of brainwaves is improved. (3) A second theory: the brain certainly has a feedback effect when subject to audio stimulation; the spectral energy of brainwaves is higher (representing an enhancement function) than an initial working state, and the spectrum energy of brainwaves is lower (promoting resting and resetting) than the initial working state. (4) A second inference: the spectral energy of brainwaves during sleep is observed, and the channel block functionality, such as transmitting, processing, life sustaining (master control), and alert zone, may be determined according to the energy spectrum subjected to the stimulation. (5) A third theory: the brain releases rest information automatically after excessive work.

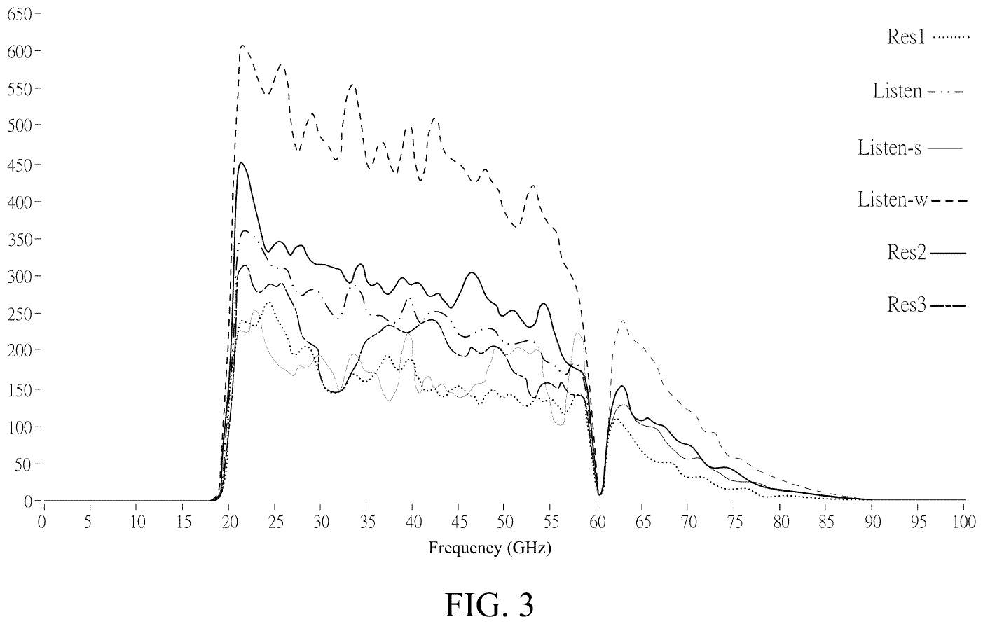

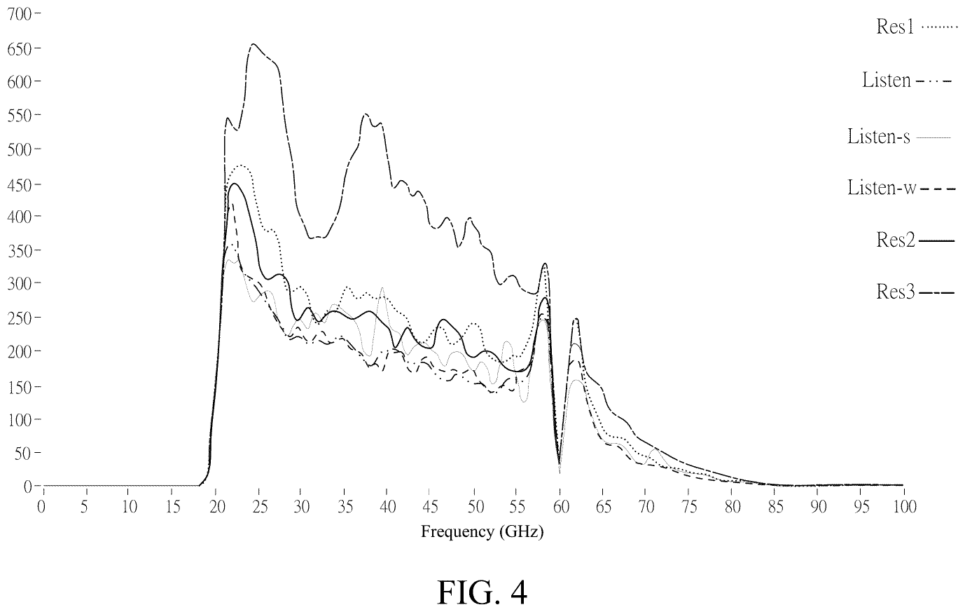

Referring to FIG. 3 and FIG. 4 for determination of the life sustaining state and transmitting and processing areas of the brain. FIG. 3 is a spectral energy diagram of brainwaves of a channel signal (auditory area T6) at different time intervals according to an embodiment of the present disclosure; and FIG. 4 is a spectral energy diagram of brainwaves of another channel signal (visual area O1) at different time intervals according to an embodiment of the present disclosure. The signal Res1 represents a signal of a rest period of the subject at a previous stage; the signal Listen represents a signal of a period during which the subject is subjected to the audio signal stimulation but is awake; the signal Listen-s represents a signal of a period during which the subject is subjected to the audio signal stimulation but falls asleep; the signal Listen-w represents a signal of a period during which the subject wakes up when the subject is subjected to the audio signal stimulation; and the signals Res2, Res3 respectively represent signals of a rest period after the subject is subjected to the audio signal stimulation. By identifying changes of the signals after the subject is subjected to audio stimulation, the functionality of the stimulated area corresponding to each channel 21 may be distinguished. For example, the auditory area T6 is a signal transmitting and processing area; and the visual area O1 is a lift sustaining signal receiving area.

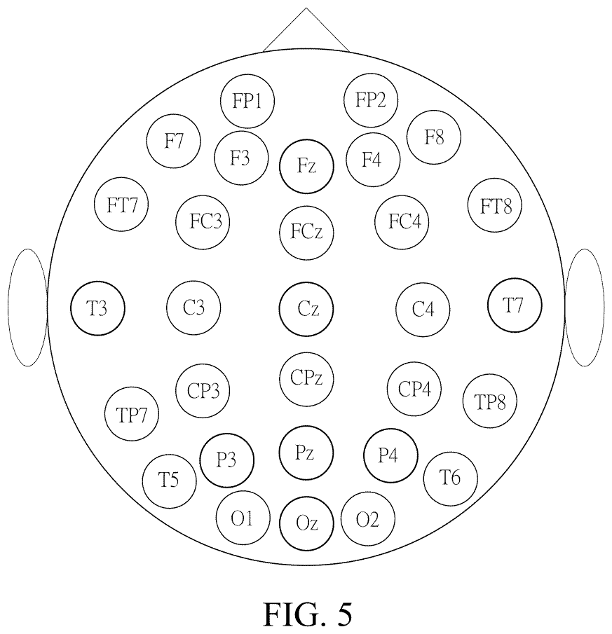

Referring to FIG. 5, which is a distribution diagram of electrodes 21 according to an embodiment of the present disclosure. Herein, 32 electrodes 21 are used. Based on the determination of the transmitting and processing areas and the lift sustaining state of the brain, signal transmission and flow direction are determined from left brain electrodes (F7, F3, FT7, FC3, T3, C3, TP7, CP3, T5, and P3) and right brain electrodes (F4, F8, FC4, FT8, C4, T4, Cp4, TP8, P4, and T6), and the operation transmission signals of the various areas of the brain are acquired. That is, the pairwise correlation is calculated from brainwave signals of the left electrodes (F7, F3, FT7, FC3, T3, C3, TP7, CP3, T5, and P3) and the right brain electrodes (F4, F8, FC4, FT8, C4, T4, Cp4, TP8, P4, and T6).

Herein, the system equation set of the areas of the 32 electrodes 21 subjected to audio stimulation is expressed by Equation 1, where F(t) is the audio stimulation, X₁-X₃₂ are the channel signals of the 32 electrodes 21, G1 is a function, which shows change in response to the channel signals from any one of the electrodes 21, U₁-U₃₂ show components independent of the channel signals, \dot{X}₁˜\dot{X}₃₂ shows the first order differential of X₁-X₃₂, and [symbol] ₁˜ [symbol] ₃₂ shows the second order differential of X₁-X₃₂.

$$( Equation 1 )$$

$$\{ \begin{matrix}{\ddot{X}}_{1} + {G}_{1} ( {X}_{1} , {X}_{2} , ... , {X}_{32} , {\dot{X}}_{1} , {\dot{X}}_{2} , ... , {\dot{X}}_{32} ) {\dot{X}}_{1} + ∇ {U}_{1} = F ( t ) \\ {\ddot{X}}_{2} + {G}_{1} ( {X}_{1} , {X}_{2} , ... , {X}_{32} , {\dot{X}}_{1} , {\dot{X}}_{2} , ... , {\dot{X}}_{32} ) {\dot{X}}_{2} + ∇ {U}_{2} = F ( t ) \\ ... \\ {\ddot{X}}_{32} + {G}_{1} ( {X}_{1} , {X}_{2} , ... , {X}_{32} , {\dot{X}}_{1} , {\dot{X}}_{2} , ... , {\dot{X}}_{32} ) {\dot{X}}_{32} + ∇ {U}_{32} = F ( t )\end{matrix}$$

In some embodiments, base frequencies of the output audio include 20 Hz, 40 Hz, and 80 Hz.

The way to analyze the pairwise correlation in the channel signals is described herein, which is applicable to the first channel signals and the second channel signals (for simplicity of description, they will be referred to as the channel signals herein). Firstly, the channel signals are converted into a plurality of time-varying frequency signals. The conversion may be done, for example, by executing a short-time Fourier transform, as shown in Equation 2. s(t) is the channel signal, and w(t) is a function of a time window.

STFT(t,f)=∫_{-∞}^{∞}w(t−τ)s(τ)e^{-j2πfτ}dτ (Equation 2)

Then, a time series of phases of the time-varying frequency signals is determined by using Hilbert transform, which has the form of Equation 3, to form Equation 4. p.v. is a Cauchy principal value, S(t) is an analysis signal, A(t) is an instantaneous amplitude, and φ is an instantaneous phase.

$$H ( s ( t ) ) = \frac{1}{π} p . v . {\int }_{- ∞}^{∞} \frac{s ( τ )}{t - τ} d τ \qquad ( Equation 3 )$$

$$S ( t ) = s ( t ) + iH ( s ( t ) ) = A ( t ) {e}^{j ( wt + φ )} \qquad ( Equation 4 )$$

After the previous processing, a pairwise phase synchronous rate in the time-varying frequency signals is analyzed. In some embodiments, the phase synchronization index (PSI) may be taken for computing. Considering the case where two coupled harmonic oscillators (a, b) have phases φ a(t) and φ b(t), respectively, the phase difference is shown in Equation 5, where n and m are ratios, which are set to be 1.

φ_{n,m(}t)=nφₐ(t)−mφ_{b}(t) (Equation 5)

Shannon Entropy is defined as Equation 6, and Smax-ln (N), where N is the number. PSI is defined as Equation 7, where 0≤\tilde{ρ}ₙₘ≤1; the larger the value of \tilde{ρ}ₙₘ is, the higher the synchronous rate of the two is; \tilde{ρ}ₙₘ=0 indicates out of synchronization; and \tilde{ρ}ₙₘ=1 indicates perfect synchronization.

$$S = - {\sum }_{k = 1}^{N} p ( k ) ln p ( k ) \qquad ( Equation 6 )$$

$${\tilde{ρ}}_{n m} = \frac{{S}_{max} - S}{{S}_{max}} \qquad ( Equation 7 )$$

Herein, a threshold may be set, and if the calculated PSI exceeds the threshold, it indicates that the phase synchronous rate is high. Therefore, the channel signals with high phase synchronous rate are used as the high-correlation response channels.

In order to study the brain response to audio signal stimulation, a test was conducted with 15 healthy subjects (10 males and 5 females) in the present disclosure. The test process was executed in the following three stages. Stage (I): the subjects closed eyes and had a rest for 3 minutes. Stage (II): the audio generation method for brain dynamics audio stimulation was executed, and the subjects were subjected to audio signal stimulation for 12 minutes, and in this period, the frequency, intensity and the like were changed. Stage (III): the subjects had a rest and closed eyes for 3 minutes. The pairwise correlation of the analyzed channel signals was performed by the brainwave signals captured in the test process to find out the output audio.

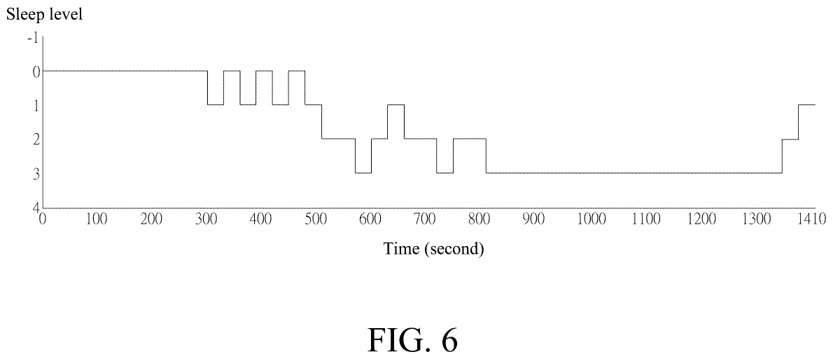

After the output audio (brain dynamics audio) was found, an effect test of the brain dynamics audio was executed. The process included the following three stages. Stage (I): the subjects closed eyes and had a rest for 3 minutes. Stage (II): the subjects listened to the brain dynamics audio music (output audio) for 18 minutes. Stage (III): the subjects had a rest and closed eyes for 3 minutes. Referring to FIG. 6, which is a schematic diagram of sleep levels of a subject subjected to audio stimulation according to an embodiment of the present disclosure. Determination of the sleep levels is based on version 2.4 of determination standard of American Academy of Sleep Medicine (AASM), sleep is divided into three levels N1, N2, N3, and the third level N3 is the deepest sleep. The subject closed eyes and had a rest for 3 minutes at stage (I) and was awake, entered stage (II) to listen to brain dynamics audio music at the 180th second, felt sleepy at the 300th second and entered the first level (N1) of sleep, entered the second level (N2) of sleep at the 500th second, and entered the third level (N3) of sleep at the 580th second or so, and afterwards, the subject slept in levels of N2 and N3 alternatively; and finally, the subject entered deep sleep N3 until the brain dynamics audio music is turned off.

According to the audio generation device and method for brain stimulation provided by some embodiments of the present disclosure, brain dynamics audio signals for specific stimulation purposes may be efficiently generated, and the outputting of the generated audio signals can effectively perform brain stimulation. A method for brain stimulation different from transcranial stimulation is provided by brain dynamics audio stimulation, which tries to make brain neuron activity to change, so as to improve symptoms, and make brain dynamics audio music treatment feasible, and have feasibility of improvement of physiological or psychological discomforts such as insomnia, anxiety, depression, and great psychological stress, or brain-related problems such as migraine and dementia.

What is claimed is:

1. An audio generation device for brain dynamics audio stimulation, comprising:
an audio playing device, configured to sequentially output multiple audio signals with multiple audio frequencies to give a subject sound stimulation;
a brainwave capturing device, configured to capture a first brainwave signal from the subject, the first brainwave signal comprising a plurality of first channel signals; and
a computing device, coupled with the brainwave capturing device, and configured to analyze pairwise correlation of the first channel signals to obtain a plurality of high-correlation first response channels corresponding to the multiple different audio frequencies, and synthesize a test audio according to at least one audio frequency corresponding to a target channel, wherein the target channel is selected from the first response channels, and corresponds to a target stimulation area; and
wherein the audio playing device is further configured to output the test audio, the brainwave capturing device is further configured to capture a second brainwave signal from the subject, the computing device is further configured to analyze pairwise correlation in a plurality of second channel signals in the second brainwave signal to obtain a plurality of high-correlation second response channels, and the second response channels comprise the target channel, and the test audio is taken as an output audio.

2. The audio generation device for brain dynamics audio stimulation according to claim 1, wherein analyzing the pairwise correlation in the first channel signals refers to converting the first channel signals into a plurality of first time-varying frequency signals, analyzing pairwise phase synchronous rate in the first time-varying frequency signals, and taking the first channel signals with high phase synchronous rate as the high-correlation first response channels.

3. The audio generation device for brain dynamics audio stimulation according to claim 1, wherein analyzing the pairwise correlation in the second channel signals refers to converting the second channel signals into a plurality of second time-varying frequency signals, analyzing pairwise phase synchronous rate in the second time-varying frequency signals, and taking the second channel signals with high phase synchronous rate as the high-correlation second response channels.

4. The audio generation device for brain dynamics audio stimulation according to claim 1, wherein the computing device is configured to determine whether the target channel belongs to the second response channels, and if the target channel belongs to the second response channels, the test audio is taken as the output audio.

5. The audio generation device for brain dynamics audio stimulation according to claim 1, wherein the computing device is configured to determine whether the target channel belongs to the second response channels, and if the target channel does not belong to the second response channels, then another test audio is synthesized according to at least one audio frequency corresponding to the target channel.

6. The audio generation device for brain dynamics audio stimulation according to claim 1, wherein base frequencies of the output audio comprise 20 Hz, 40 Hz, and 80 Hz.

7. An audio generation method for brain dynamics audio stimulation, comprising:
(a) outputting an audio signal with an audio frequency via an audio playing device to give a subject sound stimulation;
(b) capturing a brainwave signal from the subject via a brainwave capturing device, the brainwave signal comprising a plurality of channel signals;
(c) analyzing pairwise correlation in the channel signals via a computing device to obtain a plurality of high-correlation response channels;
(d) repeatedly executing steps (a) to (c) to obtain the response channels corresponding to multiple different audio frequencies;
(e) determining a target channel corresponding to a target stimulation area from the response channels by the computing device; and
(f) synchronizing a test audio according to at least one audio frequency corresponding to the target channel by the computing device, outputting the test audio via the audio playing device, and executing steps (b) and (c), and making the test audio as an output audio when the target channel belongs to the response channels.

8. The audio generation method for brain dynamics audio stimulation according to claim 7, wherein analyzing the pairwise correlation in the channel signals comprises:
converting the channel signals into a plurality of time-varying frequency signals;
determining time series of phases of the time-varying frequency signals;
analyzing a pairwise phase synchronous rate in the time-varying frequency signals; and
taking the channel signals with high phase synchronous rate as the high-correlation response channels.

9. The audio generation method for brain dynamics audio stimulation according to claim 7, wherein step (f) further comprises: if the target channel does not belong to the response channels, then synthesizing another test audio according to at least one audio frequency corresponding to the target channel, and executing step (f) again.

10. The audio generation method for brain dynamics audio stimulation according to claim 7, wherein base frequencies of the output audio comprise 20 Hz, 40 Hz, and 80 Hz.
